(19) United States
(12) Patent Application Publication
(10) Pub. No.: US 2010/0240883 A1
(43) Pub. Date: Sep. 23, 2010

(54) Lipid-drug conjugates for drug delivery

(76) Inventors: Nian Wu, North Brunswick, NJ (US); Brian Charles Keller, Antioch, CA (US)

Correspondence Address: Lee Pederson, 712 East Main Street, Sleepy Eye, MN, 56085, US

(21) Appl. No.: 12/661,465

(22) Filed: Mar. 17, 2010

Related U.S. Application Data
(60) Provisional application No. 61/210,380, filed on Mar. 18, 2009.
(60) Provisional application No. 61/217,404, filed on May 29, 2009.

(51) Int. Cl. C07C 233/38; C07D 207/46; C07H 5/06; C07H 19/06; C07D 411/04

(52) U.S. Cl. 536/28.51; 548/542; 536/55; 554/56; 544/317

(57) ABSTRACT

New prodrugs are derived from highly water soluble parent drugs that exist as primary or secondary amines in their parent form. Lipophilic carrier groups are bonded to the parent drug via an amide linkage with additional linker elements between the amide group and the carrier group.

### FIELD OF THE INVENTION

The present invention relates to drug delivery. More particularly, the present invention relates to preparing carrier-linked prodrugs from drugs having primary or secondary amine groups by using the amine group to form an amide bond between the drug and a carrier group comprised of two acyl chains.

### PRIORITY CLAIMS

This application claims priority to U.S. provisional patent application 61/210,380 filed Mar. 18, 2009 and entitled “Lipid-Drug Conjugates for Drug Delivery” and to U.S. provisional patent application 61/217,404 filed May 29, 2009 and entitled “Lipid-Drug Conjugates for Drug Delivery”.

### BACKGROUND OF THE INVENTION

Many drugs, especially oncology drugs, cannot be administrated orally due to toxicity, taste or poor system absorption and bioavailability. Therefore a parenteral administration route is the sole choice. However oral administration usually is more favorable than intravenous administration for patients. Modifying drugs into prodrugs with lipid characteristics reduces gastrointestinal side effects and improves the bioavailability of the parent drug via enhanced permeation ability of the prodrug.

Optimization timing of cleavage of prodrug conjugates for drug release is a challenge since conjugates must be sensitive enough to triggers that yield effective drug release and the triggered release mechanism should be compatible with its preexisting properties such as drug retention, circulation time, and permeation at the target sites. Research on liposomal drug delivery provides useful references regarding lipid-conjugate cleavage mechanisms. (D. C. Drummond, O. Meyer, K, Hong, D. B. Kirpotin, D. Papahadjopoulos, Pharmacol. Rev., 51 (1999) 691-743, M. B. Bally, H. Lim, P. R. Cullis, L. D. Mayer, J. Liposome Res., 8 (1998) 299-335; D. B. Fenske, I. MacLachlan, P. R. Cullis, Curr. Opin. Mol. Ther., 3 (2001) 153-158). Cleavage mechanisms can be divided into external and biological triggered systems. Heat and light are external trigging systems. pH, enzymatic cleavage or change of a redox potential are biological trigging systems.

The popularity of using esters to link parent drugs with carrier groups into prodrugs stems primarily from the fact that the human organism is rich in enzymes which are capable of hydrolyzing esters. The esterases are ubiquitously distributed and various types can be found in blood, liver, organs and tissues. By appropriate esterification of selected molecules containing an alcohol, carboxyl or amino group, it is feasible to obtain derivatives with desirable hydrophilicity or lipophilicity as well as in vivo lability. There are a great number of drugs have been modified based on alcohol and carboxylic acid using the ester prodrug approach (B. M. Liederer & R. T. Borchardt, J. Pharm. Sci. 95 (2006)1177-95).

### BRIEF DESCRIPTION OF THE INVENTION

Highly soluble drugs having primary or secondary amines are converted to prodrugs having an amide bond linking the parent drug to a lipophillic carrier group. Conversion of the drug to such a prodrug reduces gastro-intestinal side effects and increases membrane permeability. Additional functionalities included in the linkage between parent drug and carrier group allow flexibility in drug design.

### DETAILED DESCRIPTION

Embodiments of the present invention are described herein in the context of prodrugs derived from drugs having primary or secondary amine groups. Those of ordinary skill in the art will realize that the following detailed description of the present invention is illustrative only and is not intended to be in any way limiting. Other embodiments of the present invention will readily suggest themselves to such skilled persons having the benefit of this disclosure.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

Those of ordinary skill in the art will realize that the following description of the present invention is illustrative only and not in any way limiting. Other embodiments of the invention will readily suggest themselves to such skilled persons having the benefit of this disclosure.

Highly water-soluble drugs may have poor bioavailability when taken orally because their ability to cross hydrophobic biological membranes may be limited. Similarly, when delivered parenterally, they may have difficulty crossing capillary membranes. A general approach to increase the effectiveness of such drugs involves covalently linking them to a hydrophobic carrier. Several variations on this general approach are described herein. The compositions and methods all share common features. First, the parent drug contains either a primary or secondary amine group. Second, the amine group of the parent drug is used to form an amide bond to link the drug with a carrier group to form a prodrug. Third, the carrier group is hydrophobic and comprises either one or two acyl chains. The various specific approaches will be described separately.

Octanol-water partition and distribution coefficients are useful parameters for predicting whether molecules are readily able to cross biological membranes because of simple diffusion may be the primary route for drug absorption in many cases. The partition coefficient is a ratio of concentrations of un-ionized compound between the two solutions. The distribution coefficient is a ratio including all ionic and non-ionic forms of the compound at a given pH. Both coefficients are typically expressed as the logarithm of the ratio of the concentrations. Since many parent drugs and resulting prodrugs described in this disclosure have ionizable moieties, it is most descriptive to characterize the compounds in terms of distribution coefficients.

Though some membrane transport may occur from the stomach (pH between 2 to 4), membrane transport from the intestine to the blood and from the blood to the tissues and organs of the body are more important considerations. Since the pH in the intestine is usually about 7.1 and the pH of the blood is usually around 7.4, distribution coefficients used in this disclosure and attached claims are intended to be measured at pH 7.2 unless otherwise indicated. Also, the distribution coefficients are to be measured at 37 degrees C. and at normal therapeutic concentrations.

Highly water soluble parent drug compounds typically have a distribution coefficient of less than 0, i.e., more than 50% of the compound will distribute to the aqueous phase. Prodrugs of the parent drugs derivatized with acyl carrier groups will have a distribution coefficient greater than 0. Preferably the prodrugs will have distribution coefficient greater than 0.5. More preferably the prodrugs will have a distribution coefficient between about 0.5 and 3.0.

The acyl chains may be selected from the saturated lipids shown in Table 1 and the unsaturated lipids shown in Table 2. The acyl chains are typically bonded via an ester linkage, though other linkages are within the scope of the invention. When depicted in chemical structures herein as “R groups” the R group is meant to include both the acyl chain and the linkage.

TABLE 1
Saturated lipids for use in the invention:

| | | | | Melting |
|---|---|---|---|---|
| common | | | | point |
| name | IUPAC name | Chemical structure | Abbr. | (° C.) |
| Caprylic | Octanoic acid | CH₃(CH₂)₆COOH | C8:0 | 16-17 |
| Capric | Decanoic acid | CH₃(CH₂)₈COOH | C10:0 | 31 |
| Lauric | Dodecanoic acid | CH₃(CH₂)₁₀COOH | C12:0 | 44-46 |
| Myristic | Tetradecanoic acid | CH₃(CH₂)₁₂COOH | C14:0 | 58.8 |
| Palmitic | Hexadecanoic acid | CH₃(CH₂)₁₄COOH | C16:0 | 63-64 |
| Stearic | Octadecanoic acid | CH₃(CH₂)₁₆COOH | C18:0 | 69.9 |
| Arachidic | Eicosanoic acid | CH₃(CH₂)₁₈COOH | C20:0 | 75.5 |
| Behenic | Docosanoic acid | CH₃(CH₂)₂₀COOH | C22:0 | 74-78 |

TABLE 2
Unsaturated lipids

| | | Δ^{x} | |
|---|---|---|---|
| | | Location of | # carbon/ |
| Name | Chemical structure | double bond | double bonds |
| Myristoleic acid | CH₃(CH₂)₃CH═CH(CH₂)₇COOH | cis-Δ⁹ | 14:1 |
| Palmitoleic acid | CH₃(CH₂)₅CH═CH(CH₂)₇COOH | cis-Δ⁹ | 16:1 |
| Oleic acid | CH₃(CH₂)₇CH═CH(CH₂)₇COOH | cis-Δ⁹ | 18:1 |
| Linoleic acid | CH₃(CH₂)₄CH═CHCH₂CH═CH(CH₂)₇COOH | cis,cis-Δ⁹,Δ¹² | 18:2 |
| α-Linolenic acid | CH₃CH₂CH═CHCH₂CH═CHCH₂CH═CH(CH₂)₇COOH | cis,cis,cis- | 18:3 |
| | | Δ⁹,Δ¹²,Δ¹⁵ | |
| Arachidonic acid | CH₃(CH₂)₄CH═CHCH₂CH═CHCH₂CH═CHCH₂CH═CH(CH₂)₃COOH | cis,cis,cis,cis- | 20:4 |
| | | Δ⁵Δ⁸,Δ¹¹,Δ¹⁴ | |
| Erucic acid | CH₃(CH₂)₇CH═CH(CH₂)₁₁COOH | Cis-Δ¹³ | 22:1 |

Due to the relative stability amides in vivo, N-acylation of amines to give amide prodrugs has only been used to a limited extent (H. Bundgaard H & M. Johansen, J Pharm Sci., 69 (1980) 44-6). For the same reason, the utility of carbamates as prodrug derivatives for amines is also limited. Introducing an enzymatically hydrolyzable ester function into the carbamate structure may evade such problem. N-(acyloxyalkoxycarbonyl) derivatives of primary or secondary amines are likely to be transformed to the parent amine in vivo (J. Alexander, R. Cargill, S. R. Mohelson and H. Schwamm, J. Med. Chem., 31 (1988) 316-22; U. S. Gates & A. J. Repta, Int. J. Pharm., 40 (1987) 249-55). Enzymatic based hydrolysis of such ester moiety in those derivatives will lead to a (hydroxy-alkoxy)carbonyl derivative which spontaneously decomposes into parent amine via an unstable carbamic acid. Acyloxy-alkyl carbamates as shown in Table 5 and Example 11 are promising biolabile prodrugs for amino functional drugs, since they are neutral compounds and combine a high stability in aqueous solution with a high susceptibility to undergo enzymatic regeneration of the parent amine by ester hydrolysis. However, in the breakdown of prodrugs where the parent drug is a primary amine, an intramolecular acyl transfer reaction leading to N-acylation or N-methylation may compete with the regeneration of the parent drug. The intramolecular N-acylation is structurally impossible in the derivatives of secondary amines. Therefore, the utility of N-acyloxyalkxycarbonyl derivaties as prodrugs of primary amines relies on a high rate enzymatic hydrolysis to compete with the undesired intramolecular reaction (H. Bundgaard, E. Jensen & E. Falch, Pharm Res. 8 (1991) 1087-93; E. Jensen, H. Bundgaard, Acta Pharm Nord., 3 (1991) 243-7; E. Jensen & H. Bundgaard, Acta Pharm Nord., 4 (1992) 35-42; N. M. Nielsen & H. Bundgaard, J Pharm Sci., 77 (1988) 285-98; N. M. Mahfouz & M. A. Hassan, J Pharm Pharmacol., 53 (2001) 841-8; Z. Shao Z, G. B. Park, R. Krishnamoorthy & A. K. Mitra, Pharm Res., 11 (1994) 237-42; V. K. Tammara, M. M. Narurkar, A. M. Crider & M. A. Khan, J Pharm Sci., 83 (1994) 644-8, C. Yang, H. Gao & A. K. Mitra, J Pharm Sci., 90 (2001) 617-24). For this particular reason, an amino acid may be added between the lipid and the amine as a “spacer” to increase the rate of enzymatic hydrolysis and to shield or interfere with a possible intramolecular acyl transfer reaction of primary amines (H. Bundgaard & J. Moss, J Pharm Sci., 78 (1989) 122-6; A. H Kahns & H. Bundgaard, Pharm Res., 8 (1991):1533-8). Amino acids that can be preferably used as a spacer are detailed in this disclosure.

Part I: In one aspect, the invention is a method of linking diacyl carrier groups, preferably diacylglycerates or diacylglycerols, to amine-containing water soluble drugs via an amide linkage. The carrier group may be activated by reacting it with disucccimidylcarbonate (DCS).

Synthesis and activation of dioleoylglycerates is shown below in Reaction Scheme 1. The reaction scheme is applicable to carrier groups having all kinds of acyl groups.

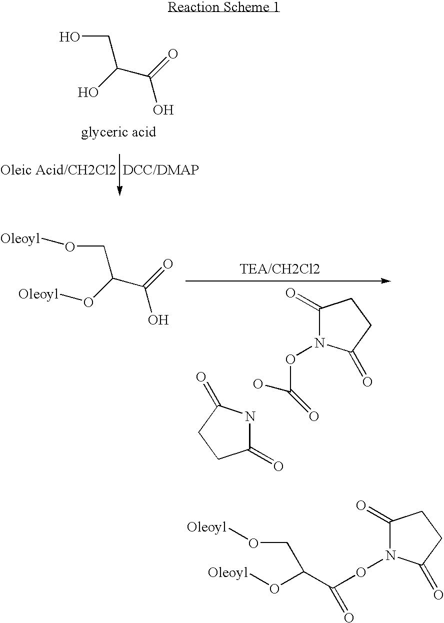

The activated diacyl carrier group may then be directly reacted with a drug having a primary or secondary amine to produce a conjugate having an amide linkage. When an activated diacylglycerate is reacted with a parent drug having a primary amine group, a prodrug as depicted in Chemical Structure 1 results. In Chemical Structure 1, R1 and R2 are acyl groups and D-HN represents the parent drug portion of the prodrug. The general structures shown in the application are meant to include all racemers and structural isomers of the structures, as they can be functionally equivalent.

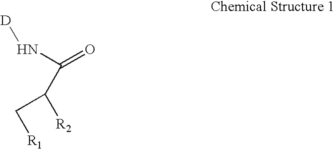

When an activated diacylglycerate is reacted with a drug having a secondary amine group, a prodrug as depicted in Chemical Structure 2 results. In Chemical Structure 2, R1 and R2 are acyl groups and D1-D2-N represents the parent drug portion of the prodrug.

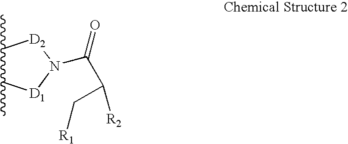

For the purposes of this disclosure, Chemical Structure 3 is meant to include both prodrugs described by Chemicals Structure 1 and Chemical Structure 2. In Chemical Structure 3, D-N(H) is the parent drug portion of the prodrug. For drugs that are primary amines in their parent form, the N atom will have a bonded H atom in the prodrug form. For drugs that are secondary amines in the parent form, there will not be a H atom bonded to N in the prodrug form.

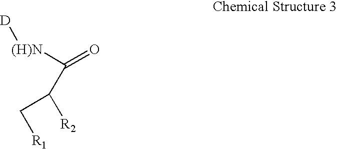

Synthesis and activation of dioleoylglycerols is shown below in Reaction Scheme 2. “Bn” indicates a benzene protective group. Again, this reaction scheme is suitable for carrier groups with all kinds of acyl chains.

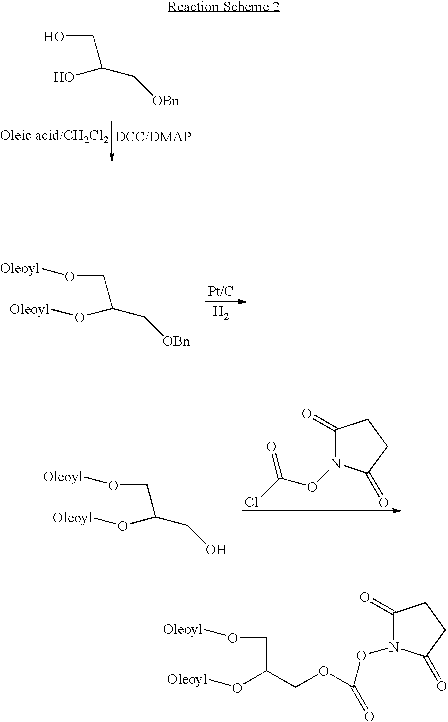

When an activated diacylglycerol is reacted with a drug having a primary amine group, a prodrug as depicted in Chemical Structure 4 results.

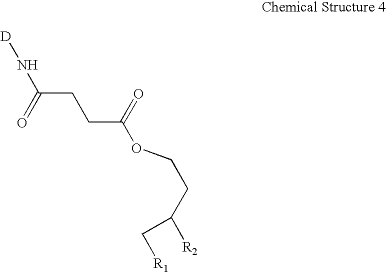

When an activated diacylglycerol is reacted with a drug having a secondary amine group, a prodrug as depicted in Chemical Structure 5 results.

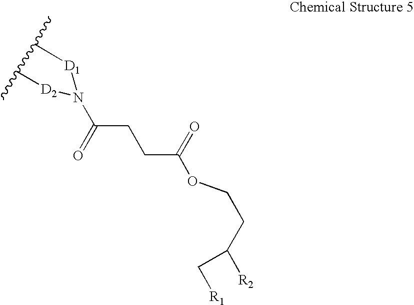

For the purposes of this disclosure, Chemical structure 6 is meant to include both prodrugs described by Chemicals Structure 4 and Chemical Structure 5.

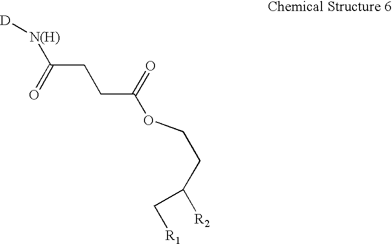

While dioleoylglycerates and dioleoylglycerols are preferred carrier groups, the approach is not limited to these. Other carrier groups having two acyl chains are also within the scope of the invention.

DCS is the preferable activation reagents used for the lipid-drug conjugation. Alternative activation reagents include not but are limited to: other analogs of N-hydroxysuccinimide, N,N′-carbonyl diimidazole or hydrazide derivatives or Schiff bases (reductive amination) or diazonium or azide derivatives or psoralen derivatives.

In one aspect, the invention is a method of linking a parent drug having either a primary or secondary amine to a diacyl carrier group to create a lipid-drug conjugate having a drug portion covalently bonded to the carrier group. The method comprises selecting a water soluble parent drug having a primary or secondary amine group; preparing a DCS derivative of a diacylglycerol or a diacylglycerate; and reacting the parent drug with the derivative to produce the lipid-drug conjugate. The parent drug has an octanol-water distribution coefficient less than about 0 and the conjugate has an octanol-water distribution coefficient greater than about 0.5. Preferably the conjugate has an octanol-water distribution coefficient between about 0.5 and 3.0. The diacyl carrier group preferably has a molecular weight between about 280 and 740. The diacyl carrier group preferably comprises two oleic acid chains.

Part II: In another aspect, the invention includes prodrugs comprised of a diacyl carrier group bonded to a drug having a primary or secondary amine via an amide bond. Such prodrugs include those shown in Chemical Structures 1 through 6.

In this aspect, the invention is a prodrug of a parent drug having either a primary or secondary amine. The prodrug comprises a diacyl carrier group linked to the parent drug via an amide linkage. The parent drug preferably has an octanol-water distribution coefficient less than about 0. The conjugate preferably has an octanol-water distribution coefficient greater than about 0.5. More preferably, the conjugate has an octanol-water distribution coefficient between about 0.5 and 3.0. The diacyl carrier group preferably has a molecular weight between about 280 and 740. The diacyl carrier group preferably comprises two oleic acid chains. The prodrug may have an ester bond between the amide linkage and the acyl carrier group. The prodrug may have an amino acid spacer between the amide linkage and the acyl carrier group. The parent drug may be voglibose.

Part III: In many cases it is desirable to insert other chemical functionalities between the hydrophobic acyl chains and the amide bond. For example, when the parent drug is a primary amine, including an appropriate amino acid spacer will help prevent acylation of the drug molecule upon hydrolysis of the amide bond in vivo. Alternatively, including an ester bond will increase the rate of hydrolysis. Including linking groups, such as amino acids with side chains shown in Table 3 or linkers shown in Table 4, provides flexibility in drug design.

In this aspect, the invention includes prodrugs according to the formula of Chemical Structure 7.

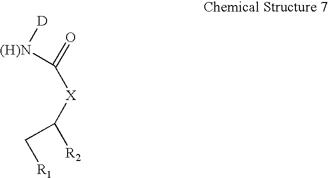

In Chemical Structure 7, R1 and R2 are acyl groups and D-N(H) is the parent drug portion of the prodrug as previously described. X may represent a variety of moieties. For example, when X is CH2, then Chemical Structure 7 is identical to Chemical Structure 6. Generally, X has a molecular weight between about 14 and 300. Specific examples are presented in this disclosure.

In one embodiment, the invention is a prodrug represented by the formula shown in chemical structure 8.

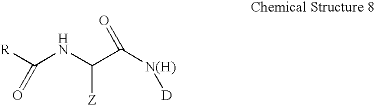

According to this aspect in Chemical Structure 8, R is a diacyl carrier group. D-N(H) represents the parent drug portion of the prodrug. Z is a side chain of an amino acid shown in table 3.

TABLE 3
Suitable amino acid spacers

| Name | Polarity | Charge at pH 7 |
|---|---|---|
| Alanine | Nonpolar | neutral |
| Cysteine | Nonpolar | neutral |
| Glycine | Nonpolar | neutral |
| Histidine | polar | positive |
| Isoleucine | nonpolar | neutral |
| Leucine | nonpolar | neutral |
| Lysine | polar | positive |
| Methionine | nonpolar | neutral |
| Phenylalanine | nonpolar | neutral |
| Serine | polar | neutral |
| Threonine | polar | neutral |
| Tryptophan | nonpolar | neutral |
| Tyrosine | polar | neutral |
| Valine | nonpolar | neutral |

In the table, amino acids containing more than one carbonyl, or both carbonyl and amide groups, are not included since the extra reactive groups may complicate the synthesis or pharmacology profile. As one of ordinary skill in the art could discern from Table 3, side chains suitable as Z in Chemical Structure 8 include: —CH3 (alanine), —CH2SH (cysteine), —H (glycine), —CH2-imidazole (histidine), —CH(CH3)CH2CH3 (isoleucine), —CH2CH(CH3)2 (leucine), —CH2CH2CH2CH2NH2 (lysine), —CH2CH2SCH3 (methionine), —CH2C6H5 (phenylalanine), —CH2OH (serine), —CH(OH)CH3 (threonine), —CH2-indole (tryptophan), —CH2-hydroxyphenyl (tyrosine), and —CH(CH3)2 (valine). Of these linkers, alanine, valine and glycine are the most preferable because they are the simplest and least polar. Lysine, leucine and isoleucine are preferable for their relative lack of polarity.

Proline may also be generally used as a spacer or linker wherever the amino acids in Table 3 are referred to in this application. When used as such, the integrity of the pyrrolidine ring is maintained. As such, prodrugs using proline as a spacer do not fit the general structures shown for using other amino acids as spacers. An exemplary prodrug using proline as a spacer is shown in Chemical Structure 9.

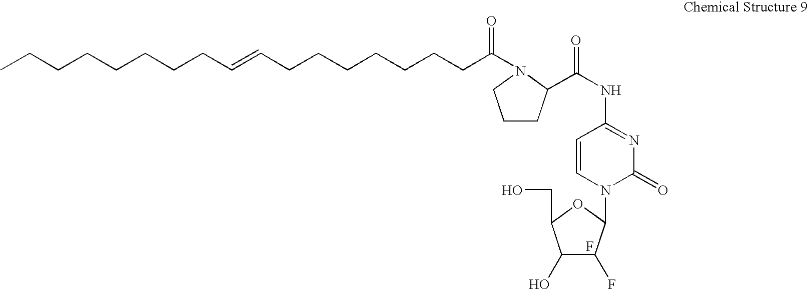

Beta amino acids may also be used as spacers, in which case an extra CH2 group would appear in Chemical Structure 8. This CH2 group would be adjacent to the carbon bearing the side chain. The same preferences for side chains exist with beta amino acids.

In this aspect the invention is a prodrug of a parent drug having either a primary or secondary amine. The prodrug represented by the formula shown at Chemical Structure 10.

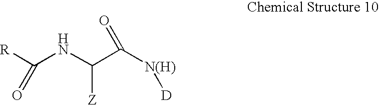

In Chemical Structure 10, R is a diacyl carrier group, N(H)-D represents the parent drug portion of the prodrug and Z is the side chain of amino acid. The parent drug preferably has an octanol-water distribution coefficient less than about 0. The conjugate preferably has an octanol-water distribution coefficient greater than about 0.5. More preferably, the conjugate has an octanol-water distribution coefficient between about 0.5 and 3.0. The diacyl carrier group preferably has a molecular weight between about 110 and 740. The diacyl carrier group may comprise two oleic acid chains. The side chain of the amino acid may be selected from table 3. The parent drug may be voglibose.

Part IV: In another aspect, the invention includes prodrugs comprised of a diacyl carrier group, a drug having a primary or secondary amine, and a non-amino acid linker between the carrier group and the drug. Such prodrugs are represented by Chemical Structure 7, where X comprises a linker selected from Table 4. The structures shown in the table were mainly named by ChemDraw. In the event of minor variations of chemical names, the structures shown are meant to be controlling.

TABLE 4
Linkers

| No | Symbol | Linker |
|---|---|---|
| 1 | N₁ | 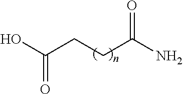 n = 1 to 18, carbamoyl-carboxylic acid |
| 2 | N₂ | 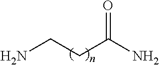 n = 1 to 18: n-amino-alkyl-amide |
| 3 | N₃ | 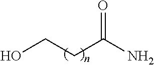 n = 1 to 18: n-hydroxyl-alkyl-amide |
| 7 | N₇ | 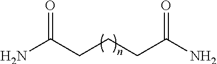 n = 1 to 18, alkyl diamide |
| 8 | N₈ | 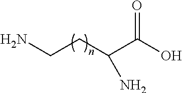 n = 1 to 18, diamino-carboxylic acid |
| 9 | N₉ | 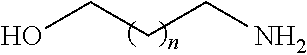 n = 2 to 18: n-aminoalcohol |
| 10 | N₁₀ | 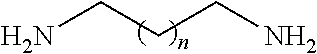 n = 2 to 18: diamine |
| 11 | N₁₁ | 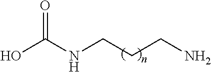 n = 1 to 18: n-amino-alkyl-carbamic acid |
| 12 | N₁₂ | 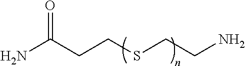 n = 1 to 12: n-amino(methyl-thio)ₙ-propanamide |
| 13 | S₁ | 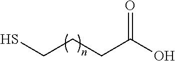 n = 1 to 18: n-mercaptocarboxylic acid |
| 14 | S₂ | 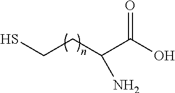 n = 1 to 18: n-mercapto-alpha-aminocarboxylic acid |
| 15 | S₃ | 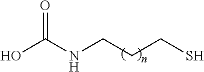 n = 1 to 18: n-mercapto-alkyl-carbamic acid |
| 16 | S₄ | 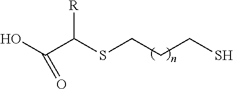 R = H or Alkyl group, n = 0 to 18 |
| 17 | S₅ | 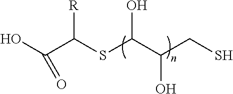 R = H or Alkyl group n = 0 to 12: n-mercaptopropylthio)carboxylic acid |
| 18 | S₆ | 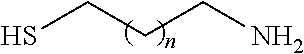 n = 1 to 18: Amino-thiol |
| 19 | S₇ | 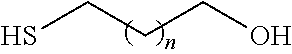 n = 1 to 18: n-mercapto-alcohol |
| 20 | S₈ | 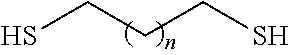 n = 1 to 18: dithiol |
| 21 | S₉ | 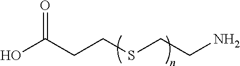 n = 1 to 18: n-amino-(methyl-thio)ₙ-propanoic acid |
| 22 | Ac₁ | 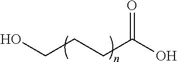 n = 1 to 18: n-hydroxy-carboxylic acid |
| 23 | Ac₂ | 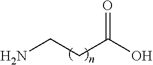 n = 1 to 18: n-amino-carboxylic acid |
| 24 | Ac₃ | 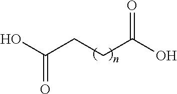 n = 1 to 18: di-carboxylic acid, n = 1: succinyl |
| 25 | Ac₄ | 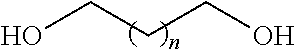 n = 1 to 18; diols |
| 26 | Ac₅ | 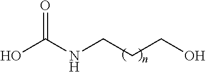 n = 1 to 18: n-hydroxy-alkyl-carbamic acid |
| 27 | Ac₆ | 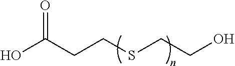 n = 1 to 18: n-hydroxyl-(methyl-thio)ₙ-propanoic acid |

In this aspect of the invention, X in Chemical Structure 7 may comprise one or more carbon atoms in addition to the linker. The linker is preferably oriented so that the carbonyl group is coupling to the drug and the amino or thiol or hydroxyl of the linker towards the lipophilic carrier group.

In this aspect the invention is a prodrug of a parent drug having either a primary or secondary amine. The prodrug is represented by the formula:

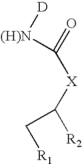

where R1 and R2 are acyl groups, N(H)-D represents the parent drug portion of the prodrug and X has a molecular weight between about 75 and 300. The parent drug preferably has an octanol-water distribution coefficient less than about 0. The conjugate preferably has an octanol-water distribution coefficient greater than about 0.5. More preferably, the conjugate has an octanol-water distribution coefficient between about 0.5 and 3.0. The diacyl carrier group preferably has a molecular weight between about 280 and 740. The diacyl carrier group preferably comprises two oleic acid chains. The linker may be chosen from those shown in table 4. The parent drug may be voglibose.

Part V: In another aspect, the invention includes a method of designing a prodrug using the linkers in Table 4. In this aspect the present invention describes new linking chemical groups that can be selected to optimize and improve lipid-drug pharmacological profile. For example, selecting an appropriate linker between a drug compound and diacylglycerol can be important for several reasons, as described below.

Since a drug is a xenobiotic, the normal human body doesn't need it. Ideally, a drug should reach the site of action intact, cure the disease, and leave the body after it completes its mission. However, drug developers often face the dilemma that a potential drug is either metabolized or excreted from the body too fast, so that the drug can not reach its site of action and achieve its therapeutic effect, or too slow, so that it stays in the body for a long time causing side effects. An object of this invention is to develop drug-lipids with unique linkers to help drugs to achieve therapeutic goals.

Similarly, different microenvironments within the body favor different breakdown processes. For example, acidic gastric fluids favors breakdown of thiol linkages. Therefore, it is still another object of this invention to provide linkers for designing prodrugs for diverse physiological microenvironments.

The method comprises selecting a parent drug with high water solubility and low lipophilicity, and having a primary or secondary amine group. A lipophilic carrier group is selected and bonded to the parent drug via an amide bond, with a linker selected from Table 4 interposed between the amide bond and the lipophilc carrier group. The resulting prodrug is represented by Chemical Structure 7, where X comprises a linker selected from Table 4. The linker is selected to provide desired stability and breakdown properties depending on the mode of administration and the target of the drug.

In this aspect, the invention is a method for making a prodrug of a parent drug having either a primary amine group. The prodrug is represented by the formula

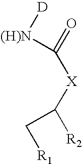

where R1 and R2 are acyl groups, N(H)-D represents the parent drug portion of the prodrug and X has a molecular weight between about 75 and 300. The method comprises selecting a water soluble parent drug having a primary or secondary amine group; selecting a linker from those shown in table 4; selecting acyl groups from those shown in tables 1 and 2; and synthesizing the prodrug. The parent drug preferably has an octanol-water distribution coefficient less than about 0. The conjugate preferably has an octanol-water distribution coefficient greater than about 0.5. More preferably the conjugate has an octanol-water distribution coefficient between about 0.5 and 3.0. The diacyl carrier group preferably has a molecular weight between about 280 and 740. The diacyl carrier group preferably comprises two oleic acid chains. The linker is chosen from those shown in table 4. The parent drug may be voglibose.

Part VI: Prodrugs having hydrophobic carrier groups comprised of a single acyl chain may also be useful in accordance with the invention. In this aspect, the invention includes a prodrug represented by the formula shown in chemical structure 11.

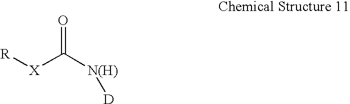

In this case, the drug has a primary or secondary amine and incorporated into a prodrug via an amide linkage. R comprises a single acyl group. X may comprise a linker selected from Table 4. Alternatively, X may comprise an amino acid linker. In the case of the amino acid linker, the invention includes a prodrug represented by the formula shown in Chemical Structure 8, where R represents a hydrophobic carrier group comprised of a single acyl chain and Z is selected from the amino acid side chains shown in Table 3. Prodrugs with extra functionality between the amide linkage and the acyl carrier group are superior to those without the extra functionality for reasons cited in this disclosure.

In this aspect the invention is a prodrug of a parent drug having either a primary or secondary amine. The prodrug is represented by the formula:

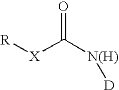

where R is an acyl group, N(H)-D represents the parent drug portion of the prodrug and X has a molecular weight between about 75 and 300. The parent drug preferably has an octanol-water distribution coefficient less than about 0. The conjugate preferably has an octanol-water distribution coefficient greater than about 0.5. More preferably the conjugate has an octanol-water distribution coefficient between about 0.5 and 3.0. The diacyl carrier group preferably has a molecular weight between about 280 and 740. The diacyl carrier group may comprise two oleic acid chains. X may comprise an ester bond. X may comprise an amino acid spacer. X may comprise a linker chosen from those shown in table 4. The parent drug may be voglibose.

Part VII: In another aspect, the invention is a prodrug of the drug voglibose. Diabetes is chronic metabolic disorder characterized by hyperglycemia which is due to relative or absolute deficiency of insulin or insulin resistance. Voglibose is an alpha-glucosidase inhibitor, used for lowering post-prandial blood glucose levels in people with diabetes mellitus. This very soluble compound causes gastro-intestinal discomfort such as flatulence, increased flatus, constipation and diarrhea [Baba S. Alpha-glucosidase inhibitor, in: Novel Development in Pharmacological Therapy of Diabetes, Baba S. Eds. Churchill Livingstone, Japan, 1994: 53-54]. These dose-related side effects sometimes result in discontinued use. By using the lipid conjugate derivative of this drug, the molecule becomes less water soluble and more lipophilic which reduces the GI side-effects.

In this aspect the invention is a prodrug of the drug voglibose comprising: a voglibose portion and an amide bond linking the voglibose to an carrier group. The prodrug preferably has an octanol-water distribution coefficient greater than about 0.5. More preferably, the prodrug has an octanol-water distribution coefficient between about 0.5 and 3.0. The carrier group preferably has a molecular weight between about 280 and 740. The carrier group may comprise two oleic acid chains.

Part VIII: In another aspect, the invention relates to prodrugs of doxorubicin. Doxorubicin is somewhat of a special case, in that it is naturally somewhat lipophilic. However, the importance of doxorubicin as a cancer therapeutic and the ability to mitigate its side effects by employing the delivery systems of the present invention warrant its inclusion in this patent. When derivatized according to the present invention and given via IV, the derivative has longer circulation, lower toxicity and an improved therapeutic profile. Also, when derivatized according to the present invention, it may safely and effectively be administered orally to a mammal. In addition to the derivatived mentioned elsewhere in this disclosure, doxorubicin may simply be derivatized by linking an acyl chain to its amine via a linkage as shown in Chemical Structure 12. Oleate and stearate are the most preferable acyl groups.

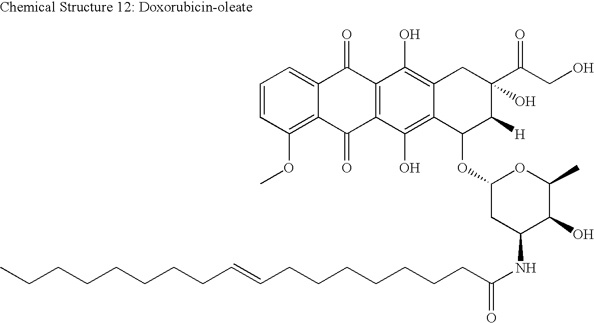

Utility of the Invention.

Two crucial factors in creating prodrugs in accordance with the present invention are the stability profiles of the prodrug in various environments, and the ability of the prodrug to regenerate the parent drug at the appropriate time and in the appropriate location.

The present invention may be used with a wide variety of drugs having either a primary or secondary amine group. The invention is particularly useful with such drugs that are both highly water soluble and highly lipophobic. In general, such drugs have a water octanol distribution coefficient (log P_{OW}) less than about 0 (negative). Adding a lipophilic carrier group to a highly water soluble drug in accordance with the invention offers several advantages. A primary advantage is improved biodistribution by providing prodrugs that are better able to cross biological membranes including the blood brain barrier than the parent drugs. In particular, oral bioavailability of many drugs can be improved by the conjugates of the invention. Another advantage is providing prodrugs with selected chemical properties to optimize stability and hydrolysis in different environments such as GI tract, bloodstream and targeted tissues.

The present invention is useful in a variety of situations, and provides advantages over conventional incorporation of drugs by lipids or polymers such liposomes in several different ways. Major obstacles for the development of liposomal formulations were—and partly still are—limited physical stability of the dispersions, drug leakage, low activity due to lack of specific tumor targeting, non specific clearance by the mononuclear phagocytic system and difficulties in upscaling manufacturing [D D. Lasic, Tibtech., 16 (1998) 307-321]. The problems with lipid-based drug formulation, liposome preparation, reproducibility, colloidal stability, sterilization, and storage may be reduced by employing the invention. For highly water soluble drugs to be well absorbed across the gastrointestinal (GI) tract and provide good bioavailability after oral dosing, a number of potentially limiting factors must be overcome. These include appropriate stability and solubility in the GI fluids, reasonable intestinal permeability, and resistance to metabolism both within enterocytes and the liver. The oral bioavailability of poorly lipophilic drugs may be enhanced in the gastrointestinal tract by this invention. Since the lipid-drug conjugates contain both hydrophobic and hydrophilic ends, they can act as a micelle to form spontaneous self suspension and monolayer or bilayer. Furthermore, the shapes of micelles or types of vesicle can be varied depends on the type of drug molecules or lipids used to form the conjugates. For example, a palmitate-glucosamine conjugate forms a suspension of linear and worm-like micelles at room temperature observed under microscope.

Unlike other lipid-based drug delivery system where the drugs are incorporated with a mixture of various lipids and other additives, the lipid-drug conjugates in the present invention are covalently bonded and thus very stable physically. The lipid-drug conjugate can be homogeneously dispersed in aqueous solutions. These lipid-drug conjugates are chemically stable in aqueous solution and can be stored at room temperature for more than two years without significant degradation.

Most conventional chemotherapy involve drug administration by injection or infusion, resulting in significant amounts of the toxic drugs in blood circulation immediately after administration and below the desired threshold concentration towards the end of the dosing interval. In contrast, oral chemotherapy can provide a prolonged and continuous exposure of the tumor cells to a relatively lower and safer concentration of the antitumor drugs. In addition, oral chemotherapy is often preferable by patients due to flexibility in dosing schedule and convenience.

The prodrugs of the present invention may provide some chemoprotectant effects in the case of parent drugs used for chemotherapy. Maximal dosing of cytotoxic chemotherapy drugs is often limited by the development of severe nonmyelosuppressive toxicities. Numerous studies have demonstrated that sulfur-containing nucleophiles can antagonize the dose-limiting effects of alkylating agents on the genitourinary tract [K L. Dechant, R N. Brogden, T. Pilkington, D. Faulds, Drugs, 42 (1991) 428-67]. For example, oral delivery of 5-fluorouracil (5-FU) has shown no improvements in overall survival rate in patients with colorectal cancer [R L. Schilsky, J. Levin, W H. West, J. Clin. Oncol., 20 (2002) 1519-1526] which may be due to the catabolism mediated by a very active enzyme of dihydropyrimidine dehydrogenase in metastatic tumors. Lipid molecules are feasible as chemoprotectants in cancer chemotherapy such as Cremophor-based paclitaxel and Phospholipid-based doxorubicin. A lipid conjugate can be utilized to improve the oral bioavailability of 5-FU, a specific formulation can be also used to further improve its activity and tolerability.

Some lipid-drug conjugates can be generally injected either intravenously, intramuscularly or subcutaneously or to the target organ. Formulations can be used for systemic body distribution with a minimum risk of blood clotting and aggregation leading to embolism. A recent study reported that lipid-based nanoparticles may be used to target both drug and biological mechanisms to overcome multidrug resistance via P-gp inhibition and ATP depletion. The study showed a significantly lowering IC50 values in P-gp-overexpressing human cancer cells with doxorubicin nanoparticles [X. Dong, C A. Mattingly, M T. Tseng, M J. Cho, Y. Liu, V R. Adams, R J. Mumper, Cancer Res. 69 (2009) 3918-26]. A lipid conjugate to doxorubicin can be a simplified and more feasible delivery vehicle for such application. In addition, the drug would circulate for longer periods of time and less accumulative on the cell membrane which reduces cardiotoxicity. Furthermore, the lipid conjugate may also prevent the interaction of doxorubicin with iron which can damage the myocytes causing myofibrillar loss and cytoplasmic vacuolization.

Drugs the my be suitable for use with this invention include nucleoside analogs as follows: Abacavir, Aciclovir, Acyclovir, Adefovir, Amantadine, Amprenavir, Cidofovir, Darunavir, Delavirdine, Didanosine, Emtricitabine, Entecavir, Famciclovir, Fosamprenavir, Ganciclovir, Idoxuridine, Imiquimod, Inosine, Lamivudine, Lopinavir, Loviride, Oseltamivir, Penciclovir, Peramivir, Ribavirin, Rimantadine, Stavudine, Tenofovir, Tenofovir disoproxil, Valaciclovir, Valganciclovir, Vidarabine, Viramidine, Zalcitabine, Zanamivi and Zidovudine. Folic acid analogs that may be used include Aminopterin, Methotrexate, Pemetrexed, Raltitrexed and Pemetrexed. Purine analogs include Pentostatin, Cladribine, Clofarabine, Fludarabine, Thioguanine, Mercaptopurine. Pyrimidine analogs include Fluorouracil, Capecitabine, Tegafur, Carmofur, Floxuridine, Cytarabine, Gemcitabine, Azacitidine, Decitabine. Anthracyclines include Daunorubicin, Doxorubicin, Epirubicin, Idarubicin, Amrubicin, Pirarubicin, Zorubicin, Mitoxantrone, Pixantrone, Valrubicin, Ifosfamide and Melphalan. Alkylating agents or other classified or nonclassified agents includes Procarbazine, Melphalan, Carmustine, Lomustine or Semustine, Fotemustine, Nimustine, Ranimustine, Streptozocin, Procarbazine, Dacarbazine, Temozolomide, Tipifarnib, Seliciclib, Tiazofurine, Tiazofurin, Celecoxib, Demecolcine, Elesclomol, Elsamitrucin, Lucanthone, Mitoguazone, Vorinostat and Mitomycin. Amino sugars or hexosamines or ketosamine and its derivatives containing at least one primary or secondary amine group include Acarbose, Bacillosamine, Voglibose, Neuraminic acid, Perosamine, Daunosamine, Desosamine, Fructosamine, Galactosamine, Glucosamine, Mannosamine and Meglumine. In addition, aminoglycosides and their derivatives include Etimicin, Framycetin, Neomycin, Gentamicin, Mitomycin, Verdamicin, Mutamicin, Sisomicin, Netilmicin, Retymicin, Kanamycin, Streptomycin, Neomycin, Framycetin, Paromomycin, Ribostamycin, Kanamycin, Amikacin, Arbekacin, Bekanamycin, Dibekacin, Tobramycin, Hygromycin B, Isepamicin, Verdamicin and Astromicin. Antidiabetic agents include Metformin, Buformin, Phenformin, Carbutamide, Glipizide, Glibenclamide, Gliquidone, Glyclopyramide, Glimepiride, Alogliptin, Linagliptin, Saxagliptin, Sitagliptin, Vildagliptin, Acarbose and Benfluorex and neurotransmitters or like include Dopamine, Norepinephrine or noradrenaline, Epinephrine or adrenaline, Octopamine, Tyramine, Serotonin or 5-hydroxytryptamine, Melatonin, Histamine, glutamate, γ-aminobutyric acid, Aspartate, Glycine, Memantine, Glutamic acid, Phenylephrine, Amphetamine, Methamphetamine, Nortriptyline, Desipramine and Amoxapine. Beta2 agonists include Salbutamol, Levosalbutamol, Terbutaline, Pirbuterol, Procaterol, Orciprenaline, Fenoterol, Bitolterol, Salmeterol, Formoterol, Bambuterol, Clenbuterol, Indacaterol and additionally, Theophylline.

While the prodrugs of the present invention are most useful for parent drugs having an octanol water distribution coefficients of less than 0, the invention may also be used with parent drugs having higher coefficients, e.g. doxorubicin. While the effect of increasing membrane permeability may not be as great with these parent drugs, other benefits including reduced toxicities will result.

Sample structures of some lipid-drug conjugates are listed in Table 5.

TABLE 5
Sample of Lipid-Drug Conjugates

| Name | Chemical Structure |
|---|---|
| Gemcitabine-dioleoylglycerol | 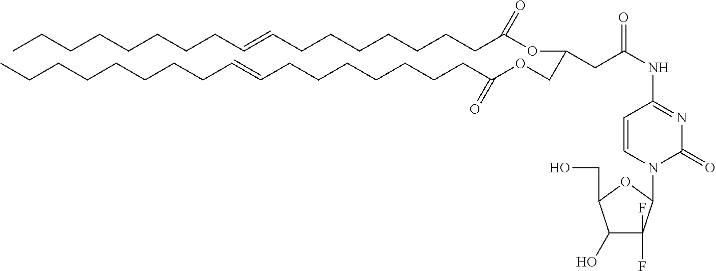 |
| Gemcitabine-cysteine-oleate | 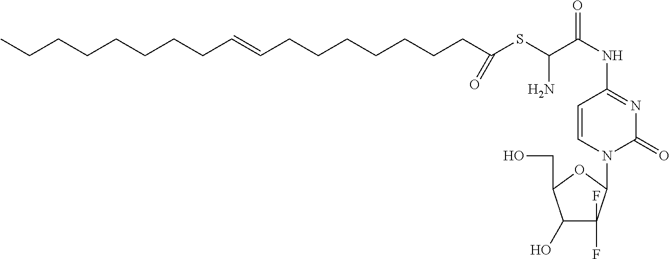 |
| Doxorubicin- succinyl- dioleoylglycerol | 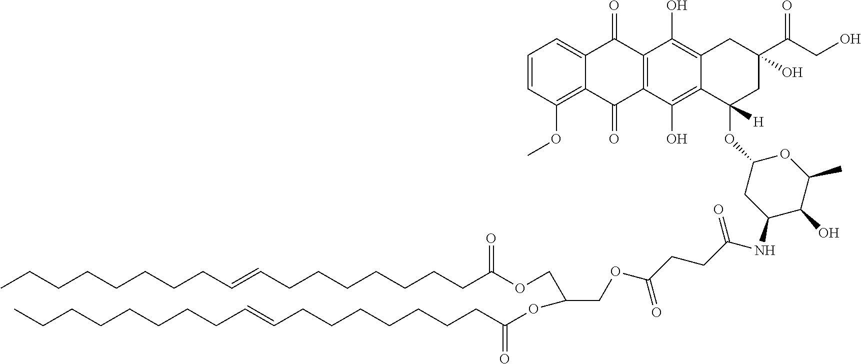 |
| Aciclovir-lysine-dioleate | 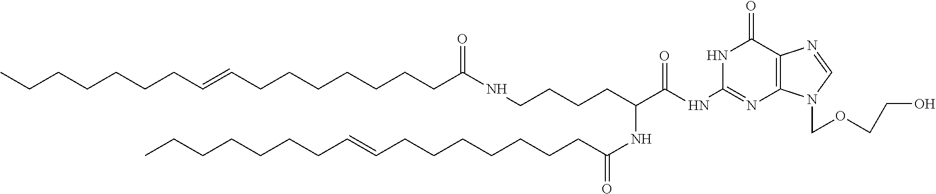 |
| Memantine-aspartate- dioleoylglycerol | 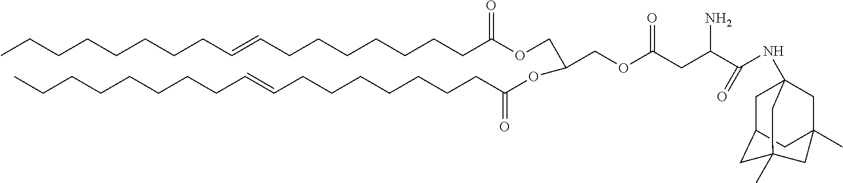 |
| Metformin-oleate | 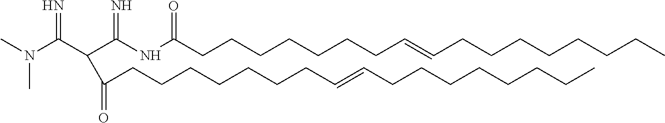 |
| Gentamicin-succinyl- dioleoylglycerol | 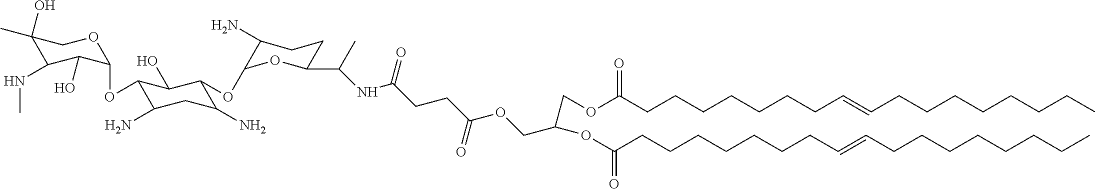 |

While embodiments and applications of this invention have been shown and described, it would be apparent to those skilled in the art having the benefit of this disclosure that many more modifications than mentioned above are possible without departing from the inventive concepts herein. The invention, therefore, is not to be restricted except in the spirit of the appended claims.

### EXAMPLES

### Example 1

### Preparation of N-hydroxysuccinimide ester of diglycerides

Disuccinimidylcarbonate (DSC) (0.15 mol) and triethylamine (0.15 mol) were added to 0.1 mole of 1,2-dioleoylglyceride, pre-dissolved in 350 mL of dimethylformamide (DMF). Stirred at room temperature for 12 h, diethylether was added, and the white precipitate was collected. The product was dispersed in ethyl acetate and left overnight in the cold. The product was filtered, washed with ether and dried in vacuo which yielded approximate 78% of the product. See Chemical Structure 13.

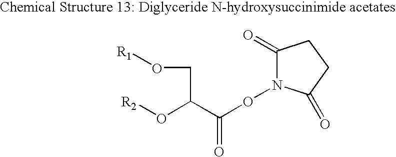

### Example 2

### Preparation of N-hydroxysuccinimide ester of diglycerol

0.1 moles of dioleoylglycerol was added in 250 mL of dried dioxane and warmed until completely dissolved. 100 mL dry tetrahydrofuran solution of 0.6 moles of N-succinimidyl chlorormate and 100 mL dry tetrahydrofuran solution of 0.6 moles of 4-(dimethylamino)pyridine were gradually added. The reaction proceeded for 3 hours under constantly stirring. White precipitate of 4-(dimethylamino)pyridine HCl and the supernatant was filtered and collected. Diethylether was added to the supernatant until no further precipitate was observed. The product was dried and stored at −20° C. See Chemical Structure 14.

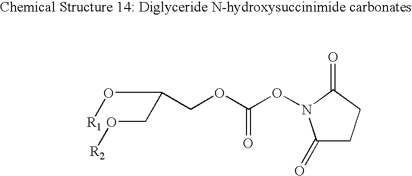

### Example 3

### Preparation of N-acylamino acids of diglyceride

A solution of N-hydroxysuccinimide ester of diglyceride or N-hydroxysuccinimide ester of diglycerol (0.1 mole) in tetrahydrofuran (250 mL) was added to a solution of L-glycine (0.1 mole) and sodium bicarbonate (1 mole) in water (25 mL). After 16 hr the solution was acidified to pH 2 with 1 N hydrochloric acid and the organic solvent was removed in vacuo. After addition of water (200 mL) the compound was filtered, dried, and crystallized from chloroformpetroleum ether to yield approximately 80% of oily product. The compound is shown as Chemical Structure 15. Other diglyceride amino acids were prepared in a similar manner.

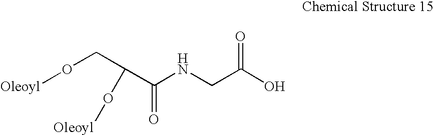

### Example 4

### Preparation of N-acylamino acids of fatty acids

A solution of N-hydroxysuccinimide ester of oleic acid (0.1 mole) in tetrahydrofuran (250 mL) was added to a solution of L-glycine (0.1 mole) and sodium bicarbonate (1 mole) in water (25 mL). After 16 hr the solution was acidified to pH 2 with 1 N hydrochloric acid and the organic solvent was removed in vacuo. After addition of water (200 mL) the compound was filtered, dried, and crystallized from chloroformpetroleum ether to yield approximately 80% of white solid with a mp of 55° C. Other oleoylamino acids or N-acylamino acids were prepared in a similar manner.

### Example 5

### Preparation of N-hydroxysuccinimide ester of 3-glycine-1,2-dioleoylglycerol

Disuccinimidylcarbonate (DSC) (0.15 mole) and triethylamine (0.15 mole) were added to 0.1 mole of N-hydroxysuccinimide-dioleoylglycerol ester, pre-dissolved in 350 mL of DMF. Stirred at room temperature for 12 h, diethylether was added, and the white precipitate was collected. The product was dispersed in ethyl acetate and left overnight in the cold. The product was filtered, washed with ether and dried in vacuo which yielded approximate 75% of the product (Chemical Structure 16).

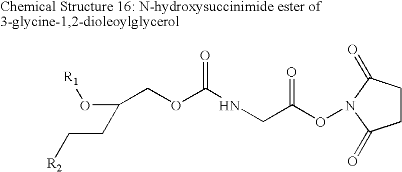

### Example 6

### Preparation of N-hydroxysuccinimide ester of oleoylamino acids

0.1 mole of glycine-oleate was added to a solution of N-hydroxysuccinimide (0.1 mole) in dry N-methyl-2-pyrrolidone (400 mL). A solution of dicyclohexylcarbodiimide (0.1 mole) in dry N-methyl-2-pyrrolidone (100 mL) was then added, and the reaction mixture was left overnight at room temperature. Dicyclohexylurea was removed by filtration, and the filtrate was concentrated under reduced pressure to yield white solid. The crude material was further purified by recrystallization from ethanol yielded approximate 87% of pure N-hydroxysuccinimide ester of oleoylamino acids, mp 49° C. See Chemical Structure 17.

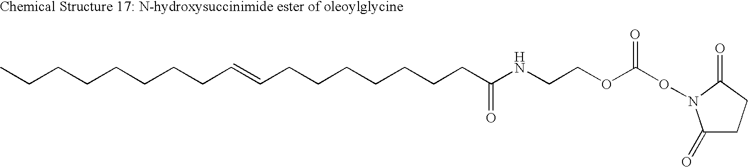

### Example 7

### Synthesis of Oleoylglycineglucosamine Ester

0.1 mole of glucosamine and N-hydroxysuccinimide ester of N-oleoyl glycine (0.11 mole) is dissolved in 200 mL of DMF and 13 mL of triethylamine (TEA) is added. The reaction mixture is stirred at 25° C. for 0.5 hr and dilute with water. The precipitate is collected via filtration and dried under vacuo. The residual is eluted in a silica gel column using a mobile phase consisting of chloroform, methanol and acetic acid (100:2:0.01). See Chemical Structure 18.

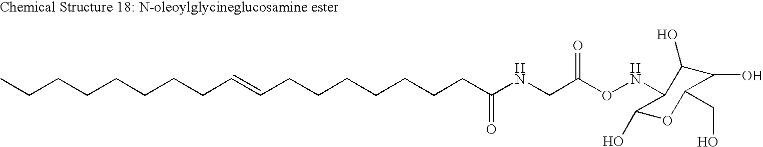

### Example 8

### Synthesis of N-oleoylglycinevoglibose Ester

0.1 mole of voglibose and N-hydroxysuccinimide ester of N-oleoylglycine (0.11 mole) is dissolved in 200 mL of DMF and 13 mL of triethylamine (TEA) is added. The reaction mixture is stirred at 25° C. for 0.5 hr and dilute with water. The precipitate is collected via filtration and dried under vacuo. The residual is eluted in a silica gel column using a mobile phase consisting of chloroform, methanol and acetic acid (100:2:0.01). See Chemical Structure 19.

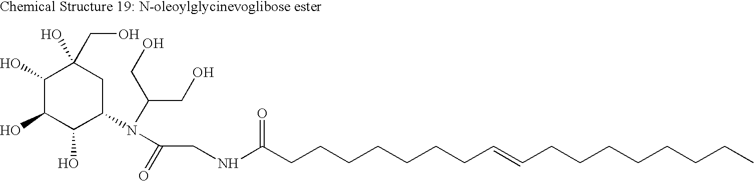

### Example 9

### Synthesis of N-oleoylglyine Gemcitabine Ester

0.1 mole of gemcitabine and N-hydroxysuccinimide ester of oleoylglycine (0.11 mole) is dissolved in 200 mL of DMF and 13 mL of triethylamine (TEA) is added. The reaction mixture is stirred at 25° C. for 0.5 hr and dilute with water. The precipitate is collected via filtration and dried under vacuo. The residual is eluted in a silica gel column using a mobile phase consisting of chloroform, methanol and acetic acid (100:2:0.01). See Chemical Structure 20.

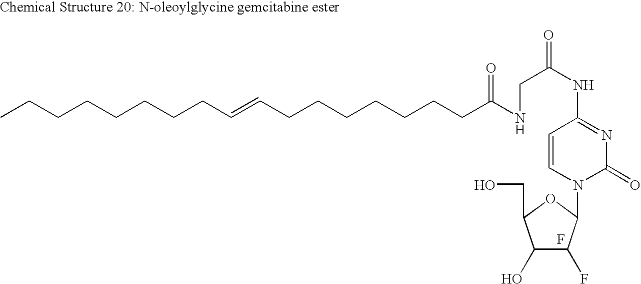

### Example 10

### Synthesis of N-oleoylglycineLamivudine Ester

0.1 mole of amivudine and N-hydroxysuccinimide ester of N-oleoylglycine (0.11 mole) is dissolved in 200 mL of DMF and 13 mL of triethylamine (TEA) is added. The reaction mixture is stirred at 25° C. for 0.5 hr and dilute with water. The precipitate is collected via filtration and dried under vacuo. The residual is eluted in a silica gel column using a mobile phase consisting of chloroform, methanol and acetic acid (100:2:0.01). See Chemical Structure 21.

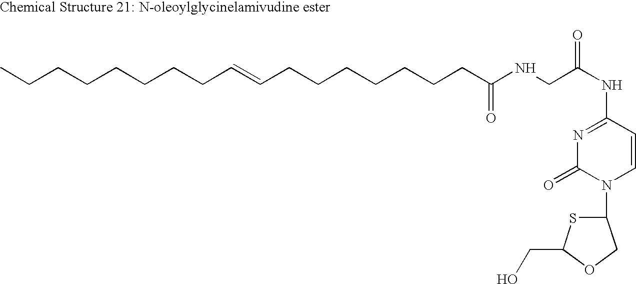

### Example 11

### Synthesis of Lipid-Drug Conjugates

Similar methods from the examples shown can be utilized for the synthesis of other monoglyceride, diglyceride and fatty acid esters of other lipid-drug conjugates. For example, see Chemical Structure 22.

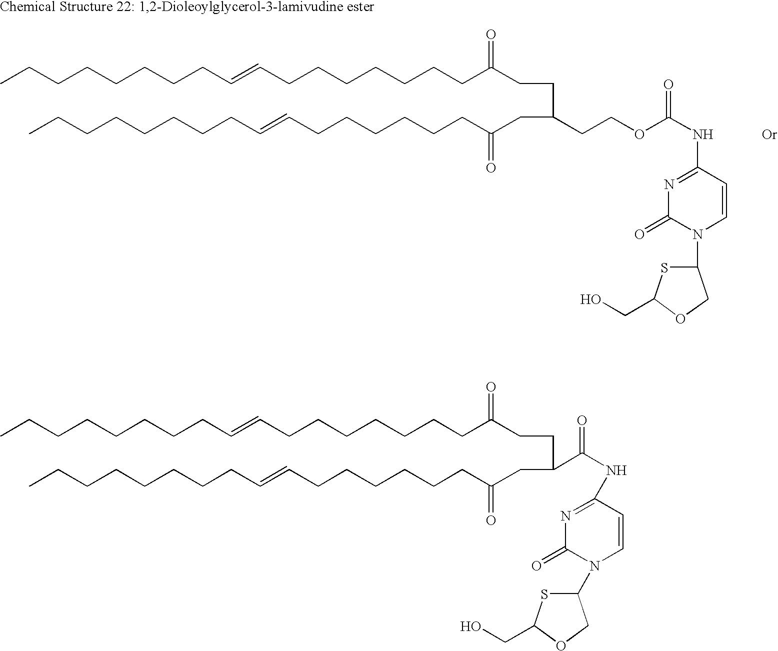

### Example 12

### Stability Experiments

Nonenzymatic hydrolysis in phosphate Buffered saline and human plasma of N-oleoyl-amino acid-lamivudine prodrugs were measured by incubating 100 to 200 μM of prodrugs in 500 μL of 10 mM KH₂PO₄ buffered saline solution (pH 7.4) at 37° C. The prodrug stock solutions were dissolved in dimethyl sulfoxide then diluted with the buffered saline solution. To determine initial reaction rates, aliquots were sampled every 30 min up to 8 hrs and quenched with TFA (1% final v/v) before being analyzed by HPLC. The estimated half-lives (t_{1/2}), obtained from linear regression of pseudo-first-order plots of prodrug concentration vs time for lamivudine prodrugs are listed in Table 6. The mass balance for prodrug disappearance and parent drug appearance was excellent (>97%). The site of esterification significantly influenced the rate of hydrolysis of amino acid ester prodrugs of lamivudine, the stability of the prodrugs in human plasma was α>β (See Chemical Structure 23 and Table 6). While the hydrolysis rates (t_{1/2}) of both L and D forms of the amino acid ester prodrugs were similar, the stability of the prodrugs at the β position was in the order isoleucine>leucine>lysine>glycine>proline>alanine.

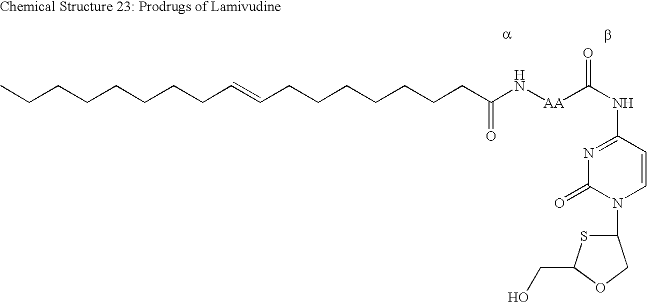

TABLE 6
Stability of Prodrugs of Lamivudine

| | t_{1/2} (min) | | |
|---|---|---|---|
| | buffered saline | | |
| | (pH 7.4) | human plasma | |
| Prodrug | β | α | β |
| L-N-oleoyl-alanyl-lamivudine | 264.0 ± 7.4 | 12.0 ± 1.1 | 5.4 ± 0.7 |
| D-N-oleoyl-alanyl-lamivudine | 277.0 ± 6.5 | 13.1 ± 1.6 | 5.6 ± 1.9 |
| L-N-oleoyl-prolyl-lamivudine | 255.0 ± 5.7 | 17.9 ± 2.0 | 6.0 ± 0.2 |
| D-N-oleoyl-prolyl-lamivudine | 274.0 ± 8.2 | 19.2 ± 1.2 | 5.9 ± 0.4 |
| L-N-oleoyl-leucyl-lamivudine | 442.0 ± 5.2 | 17.9 ± 1.4 | 5.8 ± 2.1 |
| D-N-oleoyl-leucyl-lamivudine | 452.0 ± 6.1 | 21.3 ± 1.7 | 8.1 ± 0.1 |
| L-N-oleoyl-lysyl-lamivudine | 467.0 ± 7.2 | 20.6 ± 1.3 | 8.0 ± 0.1 |
| D-N-oleoyl-lysyl-lamivudine | 452.0 ± 9.1 | 22.3 ± 2.8 | 7.9 ± 1.6 |
| L-N-oleoyl-isoleucyl-lamivudine | 461.0 ± 3.5 | 24.1 ± 2.1 | 8.6 ± 0.0 |
| L-N-oleoyl-glycyl-lamivudine | 476.0 ± 4.2 | 20.6 ± 3.3 | 7.0 ± 0.1 |
| D-N-oleoyl-glycyl-lamivudine | 483.0 ± 9.6 | 19.3 ± 2.8 | 6.9 ± 1.6 |

We claim:

1. A prodrug of a parent drug having either a primary or secondary amine, the prodrug represented by the formula: 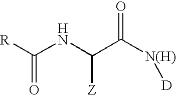
where R is a diacyl carrier group, N(H)-D represents the parent drug portion of the prodrug and Z is selected from the group consisting of —CH3, —CH2SH, —H, —CH2-imidazole, —CH(CH3)CH2CH3, —CH2CH(CH3)2, —CH2CH2CH2CH2NH2, —CH2CH2SCH3, —CH2C6H5, —CH2OH, —CH(OH)CH3, —CH2-indole, —CH2-hydroxyphenyl, and —CH(CH3)2.

2. The prodrug of claim 1, where the parent drug has an octanol-water distribution coefficient less than about 0.

3. The prodrug of claim 1, where the conjugate has an octanol-water distribution coefficient greater than about 0.5.

4. The prodrug of claim 1, where the conjugate has an octanol-water distribution coefficient between about 0.5 and 3.0.

5. The prodrug of claim 1, where the diacyl carrier group has a molecular weight between about 110 and 740.

6. The prodrug of claim 1, where the diacyl carrier group comprises two oleic acid chains.

7. The prodrug of claim 1, where the parent drug is voglibose.
